(19) United States
(12) Patent Application Publication
(10) Pub. No.: US 2005/0142220 A1
(43) Pub. Date: Jun. 30, 2005

(54) Isolated growth inhibitors in seep liquor from plants provided 3-Level nutrition

(76) Inventor: Leslie Kirkegaard, Ijamsville, MD (US)

Correspondence Address: BIOTECHPATENT. NET LLC;No. 221, 5284 Randolph Road, Rockville, MD, 20852-2116, US

(21) Appl. No.: 10/745,502

(22) Filed: Dec. 29, 2003

(51) Int. Cl. A61K 35/78; A01N 65/00

(52) U.S. Cl. 424/725; 504/117

(57) ABSTRACT

This invention provides for an odor-free run-off liquor from potted orchid plants cultivated with 3-Level nutrition possesses potent, wide-spectrum antibiotic activity. When appropriately managed, these natural growth inhibitors offers an opportunity to provide (dietary) antibiotic protection for plants and animals, including humans without selection of antibiotic resistant pathogens.

### FIELD OF THE INVENTION

This present invention relates to an isolated odor-free run-off liquor from potted orchid plants cultivated with 3-Level nutrition possessing potent, wide-spectrum antibiotic activity.

### BACKGROUND OF THE INVENTION

Soil microbes have long provided a source, and inspiration, for secondary metabolites that display useful antibiotic activity that inhibit fundamental biological processes at virtually every conceivable level of cell growth and reproduction. These metabolites, when enriched, purified, and sometimes chemically modified provide the basis for the wonder-drugs upon which human existence now depends.

It is well known that the addition of antibiotics to poultry and animal diets greatly increases outgrowth and feed conversion. Consequently, farmers seeking to produce affordable food customarily add significant amounts of selected antibiotics to the feed of their livestock. A worrisome consequence of this practice is the selection and enrichment of antibiotic-resistant pathogens that can no longer be controlled by available antibiotics. Presently, the population is faced with a choice between benefits of antibiotic use for affordable food and the creation of unstoppable pathogens.

Orchids are an example of plants that have adapted to grow in actively decaying bio-mass. Orchids have thick roots that function like an intestine that is turned inside-out so that the root takes in nutrients directly from the surrounding environment. This ability gives the orchid duel energy sources, oxidation of root absorbed compounds and photosynthesis. Efficiencies inherent in duel energy supplies enable orchids to thrive in a wide range of habitat niches.

In order to extract nutrients from the environment, orchid roots have acquired the ability to do business with soil microbes that serve a critical function in both nitrogen and carbon preservation and recycling. Soil microbiologists have long recognized the special relationship that orchid roots have with fungi. They call it mycorrhizal symbiosis¹.
1Sylivia, D., J. Fuhrmann, P. G. Hartel, and D. Zuberer, 1998. Principles and Applications of Soil Microbiology: Overview of Mycorrhizal Symbioses. Prentice Hall.

Studies to find the optimal nutrition for orchids led to the concept of 3-level feeding. 3-Level feeding provides optimal nutrients for both the plant and environmental microbes. It is formulated to provide all the minerals needed by both plant and microbes as they work on a wide variety of provided foodstuffs. It is also designed to maintain neutral environmental pH and to leave minimal salt residue after complete digestion. The formulation has been reduced to a wettable powder that is typically added to irrigation water at 1 tablespoon per gallon.

When orchids are first given water containing 3-Level nutrients, both the plant and soil microbes enter frenzied competition for nutrients. As nutrients approach exhaustion, soil microbes practice every form of chemical warfare that nature has endowed them to inhibit competitors. They do this by synthesizing secondary metabolites that serve as ‘monkey wrenches’ that clog essential reactions upon which the growth of competitors depend. Eventually a standoff is reached wherein all microbe growth is virtually prohibited. At this point the orchid becomes free to ingest the remaining nutrients supplied in the irrigation water and shifts into hyper-growth. Eventually, the buildup of natural growth inhibitors becomes so severe that even orchid growth grinds to a halt. Typically the first deleterious effect of these antibiotics on orchids is the inhibition of new buds.

Although soil microbes synthesize the antibiotic cocktail, the plant plays an important role in depleting the seep liquor of excess nutrients. After secondary metabolites have halted microbe growth, plant roots effectively ingest remaining nutrients, including nitrate. This action produces a stable, potent antibiotic product that remains tasteless, odorless and free of turbidity upon standing at room temperature. Without plants, excess nutrients in the seep liquor continue to be oxidized and rearranged by anaerobes and surviving enzymes. These reactions make the liquor distasteful upon standing.

The events described above basically characterize the wild setting for orchids. Although orchids are highly resistant to natural antibiotics they must periodically “come up for air”. Monsoon rains, winter cold and moisture, and extended drought all serve to “reset” healthy growing conditions by dissipating local antibiotic buildup.

Beautiful, exotic flowers aside, the interactions described above combine to make the orchid pot a powerful antibiotic factory, FIG. 1. When fed a wide variety of food nutrients, a competing population of microbes together with plant roots produce an equally diverse collection of growth inhibitors that are present at the lowest effective level. Amazingly the synthesis occurs odor-free and yields a product that is both tasteless and odorless. One skilled in this art, would formulate a mixture providing balanced environmental conditions in an enclosed space that enable the production of a sanitary, and uniform product.

Preparing stable, palatable seep liquor is all about adjusting the amount and concentration of 3-Level food, and the frequency of feeding such that plant and microbes completely utilize all available nutrients. Workable balance is 3-Level food at 1 tablespoon per gal applied every day or alternate days in an amount such that seep liquor is about 25% of applied food. Development of turbidity, foul odor or pH change by the seep liquor upon standing indicates residual nutrients. Healthy growth by plant indicates pot ecosystem is performing satisfactorily.

Although soil microbes synthesize the antibiotic cocktail, the plant plays an important role in depleting the seep liquor of excess nutrients. After secondary metabolites have halted microbe growth, plant roots effectively ingest remaining nutrients, including nitrate. This action produces a stable, potent antibiotic product that remains tasteless, odorless and free of turbidity upon standing at room temperature. Without plants, excess nutrients in the seep liquor continue to be oxidized and rearranged by anaerobes and surviving enzymes. These reactions make the liquor distasteful upon standing.

### SUMMARY OF THE INVENTION

The invention provides fertilizer compositions that permit experts and novices to practice 3-level plant feeding and methods of use of this preparation. At the primary level, this composition provides the minerals the plant needs for photosynthesis. At Level 2, the composition comprises a diet that supplies a variety of simple carbohydrates, aminoacids and vitamins for direct ingestion by the roots. Finally, at Level 3, the composition provides complex foodstuffs to feed a population of microbes that in turn provide a steady source of nutrients for the orchid, as well as, define the environment surrounding orchid roots. Orchid plants given balanced 3-Level nutrition grow phenomenally fast, a condition called hyper-growth. At the primary level, 3-Level nutrition provides plants with basic minerals, including nitrogen, phosphorous, and potassium, in addition to so-called, minor and trace minerals. The secondary level, provides plants with a variety of simple organic nutrients, including sugars, aminoacids, nucleic acids, fatty acids and vitamins for direct assimilation by plant roots. The third tier supplies a variety of complex carbohydrates, proteins and other food nutrients for environmental microbes, that in turn, nurture plants in several ways.

Orchids shift quickly into hyper-growth when fed daily with 3-level nutrients at 1 tablespoon per gallon of irrigation water. The amount given to each plant is such that roughly 75% is absorbed by the potting media, and 25% permeates through the pot. The odor-free seep liquor is an exceedingly powerful, yet balanced, cocktail of natural growth inhibitors formed by an extensive population of microbes fighting for limited nutrient. Antibiotic activity includes anti-bacteria, anti-fungus, anti-virus, anti-algae and anti-moss. Growth inhibitors even includes compounds that prevent plant bud formation, and consequently plays a major role in defining growing/flowering seasons for plants.

Fundamentally, seep liquor is the run-off from an enclosed, plant-microbe ecosystem optimized for the complete combustion of biomass. It is isolated as a pale yellow tea that is tasteless, meaning not salty, sweet, sour, nor bitter. Most of the time no odor is detected when the liquor is swirled in the mouth, however, depending on mouth sensitivity, it sometimes has a discernible earthy bouquet. These properties enable seep liquor to be utilized in a variety of agriculture and domestic applications.

### BRIEF DESCRIPTION OF THE DRAWINGS

FIG. 1 shows the application of preparing a stable seep liquor.

FIG. 2 shows the Orchid growing room where seep liquor is collected from table top

FIG. 3 shows the experimental medicine factory built for the production of antibiotics.

### DETAILED DESCRIPTION

In the present invention, this novel fertilizer composition and three-level plant feeding provides numerous and significant benefits for many plants. The phenomenon applied to orchids illustrates the invention.

The invention is drawn to:

Planning 3-Level Fertilizer for Pot Plants

A fertilizer that feeds plants while simultaneously feeding microbes for the insitu synthesis of balanced, slow-release nutrients offers significant advantages beyond current technology. The following disclosure teaches how to construct a powdered fertilizer formulation used at 1 tablespoon per gallon applying the science described above.

It is important to note that the described formulation goes far beyond the simple ratios of elements provided by most fertilizer preparations. Because pH and salt content are critical parameters, it provides the exact chemical species used in a balanced formula. Using the principles disclosed, one skilled in chemistry and biochemistry can formulate any number of combinations to achieve the same end-point.

Step 1, Phosphate. Start with an amount of Phosphate that can provide adequate pH buffering. For a nominal 100 grams of fertilizer, at least around 5 g of monobasic Calcium salt of Phosphate, commonly labeled Super Triple Phosphate. A working range of 5-35 grams of monobasic Calcium salt of Phosphate, preferably 10 g of monobasic Calcium salt of Phosphate can be used. This provides the principal acid component in the formulation.

Step 2, Potassium Bicarbonate. For a nominal 100 grams of fertilizer, at least around 4 gram Potassium Bicarbonate is added. This amount when hydrolyzed by Bicarbonate Dismutase balances the buffering capacity of the Phosphate. A working range of 1-10 grams of Potassium Bicarbonate, preferably, 4 gram Potassium Bicarbonate can be used. This also maximizes the Bicarbonate concentration to enhance CO₂ uptake by plant roots.

Step 3, Nitrogen Sources. For a nominal 100 grams of fertilizer, at least around 14 grams of urea, plus equal moles of Ammonium sulfate, 3.0 g, and Sodium Nitrate, 2.5 g is added. (Use of additional Ammonium Sulfate or Sodium Nitrate are potential possibilities to regulate final pH in the soil ecosystem.) These Nitrogen sources provide immediate nitrogen for plant and microbe uptake. A working range of 7-30 grams of urea; preferably at least 14 grams of Urea can be used. Use of Urea results in a minimal net contribution to residual salt build-up.

Step 4, Potassium. For a nominal 100 grams of fertilizer, at least 4 grams of Potassium Chloride is added. This amount plus the Potassium from K₂HCO₃, Step 2, equals 1 Potassium per Phosphate. A working range of 2-10 grams; preferably 4 grams of Potassium Chloride can be used.

Step 5, Citric Acid. For a nominal 100 grams of fertilizer, at least around 1.00 g citric acid is added. This amount serves to chelate Calcium and provides a starting pH near 6.5. Citric Acid serves as a regulator for the Urease and Carbonic Anhydrase enzymes. Its level determines the rate and extent of pH shifts caused by Bicarbonate and Urea concentrations. Note, upon complete oxidation, citric acid leaves no residual acid in the environment. A working range of 0.6-8.0 grams of citric acid; preferably 1.00 g citric acid can be used.

Step 6, Microbe nutrients. For a nominal 100 grams of fertilizer, at least around 6.5 grams sucrose, corn flour—around 9 g, whole-wheat flour (or an alternate energy source)—around 9 g, yeast food—around 12 g, Purina Animax-around 12 g, vegetable oil—around 5 g, and non-fat dry milk, preferably 9 g/100 g of fertilizer. These nutrients provide a wide range of carbohydrates, proteins, fats and vitamins that sustain a broad population of microbes that are unlikely to be plant pathogens. Plant roots can directly absorb many of these nutrients. Any balanced combination of agricultural plant and animal byproducts can serve as microbe nutrient. The objective is to provide a broad range of carbohydrates, proteins and vitamins, that will support a diverse population of microbes. Ideal carbohydrate sources do not support microbes that have the capability to digest plant cell walls.

Step 7, Optional pH indicator. For a nominal 100 grams of fertilizer, at least around 0.1 g Phenol Red is added. The pH indicator permits the user to visually monitor the pH of the seepage that comes out of the pot after watering. If seepage is clear/yellow, the pH is below 7. If seepage is red, the pot is dangerously alkaline, and immediate steps should be taken to lower it.

As far as possible, the above ingredients are blended in a flour-mill. Even when finely ground, the flours in the fertilizer result in a suspension when placed in water. Insoluble flours in the fertilizer solution are of no concern because the flours serve as microbe food that will eventually be solublized by microbial enzymes.

The above formulation is well balanced to maintain soil pH near 7. When added to irrigation water at 1 tablespoon per gallon, the pH of the fertilizer solution is below 6.5. When applied to soil, it drops the pH below 7. As the microbes begin digesting their nutrients, and the Anhydrase/Dismutase enzyme begins to work on the added Bicarbonate, the pH slowly increases to about 7.4. After a few days, the pH very slowly retreats toward 7. The exact pH profile depends upon local environmental parameters.

If for some reason the terminal pH needs to be adjusted, this may be done changing the amount of bicarbonate in the fertilizer preparation. Additional Bicarbonate will make the final pH higher, and less Bicarbonate will make it lower.

Effective 3-Level Feeding

The manner that plants are provided with 3-level nutrition is nearly as important as the composition of the fertilizer. Although 3-lever fertilizer is more forgiving than conventional mineral fertilizers, it still remains important to apply the fertilizer consistent with the natural cycles of the plant.

The formulation described above was designed for use at 1 tablespoon per gallon. Typically, each plant is given enough water-fertilizer to soak the pot without excessive run-off. Surfactants in the fertilizer enable the fertilizer solution to rapidly wet the soil, even when applied to dry pots. Although minimal run-off will eventually cause the build-up of excess salts by evaporation, experience to date indicates that most plants do well if flushed once a month with pure water.

Because 3-Level nutrition includes feeding environmental microbes it often proves helpful to feed plants held dormant by inclement weather. Although photosynthesis may be dormant, underground roots continue to absorb nutrients for metabolism. In addition, soil microbes capture available nutrients and incorporate them into cells that later become packets of slow-releasing fertilizer. However, sometimes plants go dormant due to diseased or salt/pH-injured roots. If these plants are given nutrition, it will generally hasten root loss and plant death.

The described fertilizer composition is not recommended for foliar feeding. Although the fertilizer contains many nutrients that are directly assimilated by leaves, the insoluble flours may leave a white residue on the leaves. It addition, it is not recommended to permit nutrient solution to stand in the crown of plants where growing microbes can infect the plant. On the other hand, the microbes supported by the fertilizer nutrients generally do not infect plants. Although the inventor has sprayed many plants with nutrient solution, he has yet to document disease caused by fertilizer standing in the crown of plants. It is yet too early to rule out that water-fertilizer standing in the crown will not propagate plant disease.

Plants given 3-Level nutrition generally shift gears into rapid growth. Although it is fun to watch plants set on new leaf and root growth, if flowers are the objective, it may be necessary to take away the 3-Level nutrition. If 3-level nutrition is stopped, the pH of the soil environment will gradually drop as residual carbon foods are metabolized and as buffering Phosphate and Carbonate salts are eluted from the soil. This pH transition is often very beneficial in jolting plant into a phase of growth.

Preferred Embodiment

The following formulation teaches one embodiment of this invention. Those skilled in biochemistry and nutrition can modify the formulation disclosed in numerous ways to achieve equivalent performance.

TABLE 1
Recipe for 3-Level plant food.

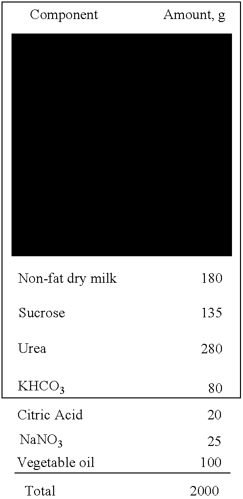

Items highlighted in dark gray are combined and ground in flour mill, Items in light gray are mixed and pulverized in food blender. After pulverizing, blue and red items are throughly mixed, and the oil is slowly added Mixing is continued until product is homogeneous.
*Yeast Food is animal feed supplement.
**Animax is multi-purpose animal food from Purina Mills LLC.

Yeast food available from Diamond Mills Inc., Cedar Rapids, Iowa, 52407, contains Saccharomyces Cerevisiae yeast and the media on which it is grown consisting of ground yellow corn, hominy feed, corn gluten feed, wheat middlings, rye middlings, diastatic malt, and corn syrup and cane molasses. It is an animal feed supplement obtained as a byproduct of alcohol production. Yeast food is added as a source of bio-nutrients, as well as, trace minerals.

Animax is a pelleted feed product by Purina Mills recommended for cattle, swine, and poultry. It is a balanced formula that is antibiotic free and contains plant and animal proteins along with vitamins and trace minerals.

The monobasic salt of Calcium Phosphate is sold as triple super phosphate.

Potassium Chloride is usually sold as potash and sometimes identified as muratic salt.

Procedure:

Combine the Group 1 ingredients together and grind in a flour-mill. Combine Group 2 ingredients together and homogenize in a mechanical mixer.
Combine products from Group 1, 2 and 3 and mix thoroughly in a mechanical mixer. The final fertilizer product is a dust-free powder.

For use, combine fertilizer product with irrigation water at a rate of 1-tablespoon fertilizer per gal water. Apply to pot plants until wet.

### Managing an Orchid Pot for Production of Growth Inhibitors

Orchids are typically planted in some kind of media consisting of large particles of slowly decaying organic material. Bark medias are particularly common. When orchids are supplied with the essential minerals necessary to support plant growth, a population of microbes will eventually be isolated that have the ability to utilize the media particles for food. This action causes the media to break-down and become toxic to orchid roots. Orchid growers manage this problem by periodically repotting their plants.

Supplying orchids with 3-Level nutrition alters this basic scenario. A complete diet both feeds and medicates the orchids to achieve hyper-growth even as media break-down is inhibited. Consequently the type of media particles become relatively unimportant, as long as, it provides a range of environmental niches extending from anaerobic to aerobic. This feature enables a diverse population of microbes to thrive.

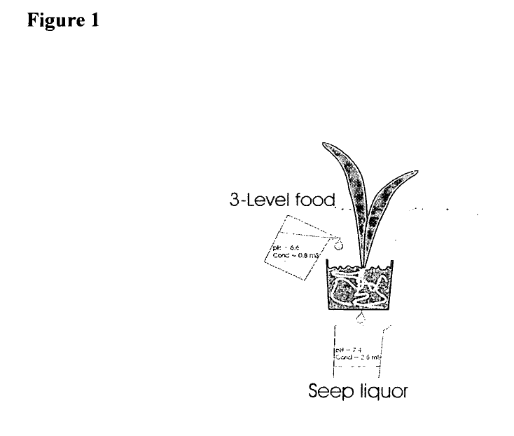

Conditions appropriate for antibiotic production are those that are optimal for orchid growth, FIG. 1. Basically, it involves the periodic watering of the orchid plant with water containing 1 teaspoon 3-Level fertilizer per gallon. A quantity is added to each plant such that the run-off amounts to approximately one-quarter of added volume.

The seep liquor consists of collected run-off water. The only treatment, if necessary, consists of permitting particulate matter to settle. A sterile filtration of product may be desirable in applications where it is essential to avoid transmission of pathogenic spores.

Usually it takes 1-2 weeks for microbes and the orchid to achieve a dynamic balance with the added nutrients.

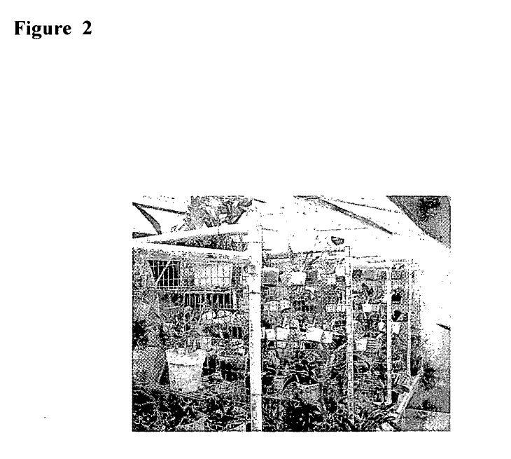

FIG. 2 shows the inventor's arrangement for growing orchids under fluorescent lights. Any lighting and atmospheric conditions compatible for growing plants is acceptable for antibiotics production.

If antibiotic production, rather than flowers is the objective, the ideal orchid is one with extensive deep root systems. Cymbidiums and like orchids are particularly excellent. Although this discovery was as an outgrowth of the pursuit of optimal growing conditions for orchids, any deep-root plant that has high resistance to soil antibiotics could work equally well. Day Lilies and related plants that respond well to 3-Level fertilizer are likely candidates for antibiotic production.

The working concept for making antibiotic is adjusting the amount of food nutrients provided in the irrigation water to balance the oxygen transfer capacity provided by the soil character. Optimal antibiotic production requires that essential mineral nutrients, including oxygen, be adequately available until all microbial growth is halted by antibiotic buildup and nutrient depletion. The idea is for nutrients to run out concurrently with antibiotic reaching plateau levels. Additionally environmental pH and salt levels must be kept within microbe sustaining levels.

An attempt to produce antibiotic in a bed of wood mulch without plants led to a less desirable antibiotic product that possesses significantly more taste and odor. This occurs because it is difficult to balance food nutrients at the point where they become depleted simultaneously with antibiotic reaching end-point levels.

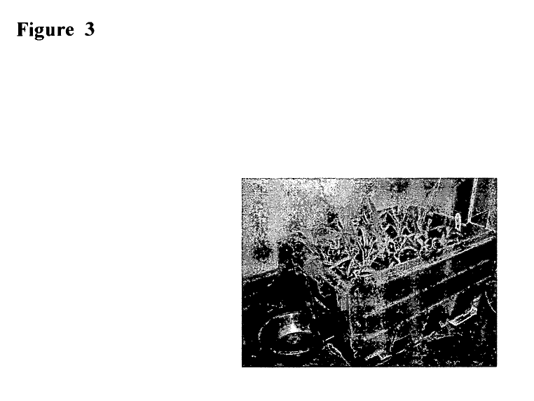

Excess foods continue to be metabolized by released enzymes and anaerobic organisms to give distasteful by-products. For many applications this potent, albeit inferior, product is quite satisfactory, particularly if it is to be used immediately. FIG. 3 is a photo of the same system after fast growing orchid seedlings have been planted. The tote bin is filled with mulch (1:1 ratio of pine bark ‘mini nuggets’ and shredded cypress mulch) into which rapidly growing orchid hybrid (Coch. Overbrook×Bollea equadoriana) seedlings have been planted. It produces about 500 ml of seep liquor when supplied with 1200 ml of water containing 4 g of 3-Level plant food.

An important outcome in the production of a comprehensive cocktail of growth inhibitors through balanced, 3-Level, plant nutrition is the impossibility of developing antibiotic-resistant microbes. A broad-spectrum of food nutrients supplied to microbes living in micro-niches provided by cavities in orchid media that range from anaerobic to direct air exposure, ensures that all possible environmental organisms will grow. In the presence of excess carbon nutrients all possible growth inhibitors will be produced to an effective concentration. Practically, there is no way for an organism to mutate sufficiently to overcome the antibiotic cocktail. Over time nature has found compartmentalization to be a way to overcome microbe produced growth inhibitors. The basic advantage of multicellular organisms arises from the ability of these organisms to restrict environmental microbes and resultant growth inhibitors to digestive and respiratory tracks and away from sensitive reproductive tissues. Basically, this is the mechanism that enables orchids to ingest available environmental nutrients after secondary metabolites have halted microbial growth. Since the orchid growth conditions for production of growth inhibitors mimics the natural setting that has been operational for eons, it is unlikely that either the production, or use of natural growth inhibitors will generate pathogens resistant to essential therapeutic drugs.

Soil insects are always a persistent problem when growing orchids or working with a bed of mulch. When necessary soil insects are controlled by watering the pot with a low residue insecticide, which are commercially available. When this treatment is performed, the orchid pot must be flushed for several days before a potable antibiotic product can again be collected.

### Applications for Natural Growth Inhibitors

Although the seep liquor is a cocktail of powerful growth inhibitors that obstruct essential life processes at virtually every level such that it brings the growth of living things to a halt, the antibiotics can not completely sterilize the liquid. This means that as the seep liquor becomes diluted, life for microbes can resume. Living forms can become separated according to their ability to resist natural antibiotics. Ultimately, in nature, ecosystems reflect the ability of life forms to break through natural antibiotic barriers. This basic fact must always be kept in mind during the production and use of seep liquor.

Plant Protection:

In the greenhouse, seep liquor antibiotics can provide essential protection from the transfer of fungal, bacterial, and viral diseases between plants. The growing room shown in FIG. 2 shows orchids hung such that the effluent coming out of upper orchid pots drips on lower plants. This notoriously bad positioning due to disease transfer is not problematic as long as plants are given 3-Level feeding that leads to endogenous antibiotic buildup. If for some reason 3-level feeding is replaced by simple watering, one can begin to find evidence for bacterial and fungal disease transfer after one week. This observation suggests that natural growth inhibitors have the potential to protect plants from fungal and bacterial diseases.

In the green-house, the antibiotic activity in the seep liquor effectively keeps walls and the surfaces under the plant pots clean and free of algae and moss.

Animal Protection:

Oral Hygiene:

Seep liquor provides a delightful alternative to chemical-based, antiseptic mouth washes. The tasteless, ordorless liquid fights oral microbes far more effectively than chemical astringents. Personal experience over several weeks suggests that rinsing the mouth with natural growth inhibitors before and after brushing and flossing proves to be a powerful constraint on plaque-forming and odor-causing bacteria.

The potent benefits of seep liquor were first discovered when inventor tested the liquid for treating an acute sore throat, the precursor of a fall cold. This involved gargling hourly over the course of the day. By the next morning all traces of a sore throat and sinus infection were gone. A single gargle treatment of the characteristic cold-sores that erupted the next day completely relieved all symptoms. Not since the miraculous recovery from a strep infection by a penicillin injection experienced “in the 50's” has the inventor encountered as dramatic a disease turn-around.

Digestion Aid:

It has long been known that livestock and poultry have much better conversion of feed to body weight when antibiotics are added to their diet. This fact provides strong incentive for food producers to supplement diets with antibiotics, a practice that eventually cultivates drug-resistant super-pathogens that no longer can be controlled by available antibiotics.

In the absence of farm animals, the inventor decided to test the effect of seep liquor on his own digestive track and later on that of “Nellie” his 16-year old fox terrier.

The first encounter involved the addition of 160 to 200 ml of seep liquor to an equal amount of home-made wine taken at bed-time. The immediate result, complete suppression of persistent gastric reflux that had long been accepted as “normal”. No sign of indigestion or of other ill effects were observed. A second test the next evening confirmed the suppression of gastric reflux; however, a slight discomfort in the “pit of the stomach” occurred.

Subsequent, more refined observations indicate that roughly 60 to 120 ml seep liquor per day, usually taken with oral hygiene, to be sufficient to inhibit gastric reflux. In addition it very effectively relieves the consequences of over-eating and the run-away gastric fermentations that give rise to sour stomach and intestinal gas.

Basically, this personal experience indicates that dietary antibiotics improve feed conversion by giving a healthy digestive system more time to extract nutrients before they are captured by gut bacteria. Dietary growth inhibitors also tilt the stand-off between gut organisms and intestinal immune responses in favor of the digestive system.

At the advanced age of 16, dietary antibiotics serve additional purposes for Nellie the fox terrier. Loss of appetite and night-time incontinence are conditions the veterinary has previously treated with antibiotic injections and tablets. These conditions are effectively managed with 30-60 ml of seep liquor per day added to drinking water or used to soften dry food.

These applications consistently indicates the broad applications of natural growth inhibitors as a dietary supplement in animal and poultry feed.

Topical Applications:

Seep liquor growth inhibitors have proved extremely effective for the treatment and cleansing of a variety of skin injuries, including cuts, scratches, abrasions, and blisters.

1. A method of making a growth inhibitor from the run-off of 3-level nutrient administered to mulch or an actively decaying biomass or plant soil.

2. The method of claim 1, wherein the plant is a deep rooted species.

3. The method of claim 2, wherein the plant is a Lily or Orchid.

4. The method of claim 1, wherein the growth-inhibitor has a potent, wide-spectrum antibiotic activity.

5. The method of claim 1, wherein the growth inhibitor activity is selected from the group consisting of anti-bacterial, anti-fungus, anti-virus, anti-algae, anti-moss.

6. Isolated growth inhibitors from seep liquor of a 3-level nutrient administered to mulch or an actively decaying biomass or plant soil.

7. A method of protecting plants from the transfer of diseases between plants comprising the composition of claim 6.

8. The method of claim 7, wherein said protection is against fungal, viral and bacterial diseases.

9. A method of protecting animals, including human beings against infections comprising the composition of claim 6.

10. The method of claim 9 wherein the protection is against skin injuries, cuts, scratches, abrasion or blisters.

11. A method of aiding digestion in animals, including human beings comprising the composition of claim 6.
